(19) United States
(12) Patent Application Publication
(10) Pub. No.: US 2010/0310054 A1
(43) Pub. Date: Dec. 9, 2010

(54) TABLE TOP FOR RADIATION THERAPY

(75) Inventor: Kjell Benny WESTERLUND, Uppsala (SE)

Correspondence Address: YOUNG & THOMPSON, 209 Madison Street, Suite 500, Alexandria, VA, 22314, US

(73) Assignee: ONCOLOG MEDICAL QA AB, Uppsala (SE)

(21) Appl. No.: 12/795,923

(22) Filed: Jun. 8, 2010

(30) Foreign Application Priority Data
Jun. 8, 2009 (SE) 0950420-0

(51) Int. Cl. A61B 6/04

(52) U.S. Cl. 378/209

(57) ABSTRACT

A radiotherapy treatment table top device (1) with high rigidity and load carrying capability, while at the same time providing a low attenuation of radiation, includes a core (2), preferably made of a foamed material, at least partly enclosed by a skin (3,3′) that preferably is made of a fiber material, whereby the core (2) is sandwiched between the skin (3,3′) on opposite sides (6,7) of the table top device (1). The core (2) is non-homogenous with respect to density. Preferably the core has a varying density, with higher density close to at least one interface (8. 8′) with the skin (3), such as a higher density in an upper layer (4) and a lower layer (5) close to the skin (3,3′) and lower density towards the central plane of the core (2).

### TECHNICAL FIELD OF THE INVENTION

The present invention relates to a table top for radiotherapy, and in particular the invention relates to a table top adapted to provide low radiation attenuation while exhibiting high stiffness.

### BACKGROUND OF THE INVENTION

As is known in the prior art, during radiation therapy or diagnostic imaging a patient is placed on the treatment table top 1 of a patient support system and a therapeutic or diagnostic beam from a radiation source 16 is projected through the patient and contacts at least a portion of the table top 11. As illustrated in FIG. 1, the patient support system 10 typically further comprises a carriage 12, which provides motorized longitudinal and lateral movement of the table top 1, a variable height arm 13 and a turntable 14. This allows the patient to be supported in a prescribed position during treatment.

In radiation therapy systems today, the treatment table top, upon which the patient is placed, absorbs a substantial fraction of the therapeutic radiation when the radiation comes from below, since the table top is made of dense materials. These table tops have to be thick in order to fulfill the stringent requirements on the rigidity of the table top, necessary in order to keep the patient in the prescribed position during treatment.

In the current technology, the attenuation of the radiation by radiotherapy treatment table tops has been limited by using a table top comprising a framework, typically made of metal. While attenuation is reduced due to open areas between the metal struts of the framework, the metal struts of the framework often interfere with the treatment, and, in addition, the amount of support received by the patient is dependent on the position of the patient with respect to the framework. To improve the patient support and to limit the interference with the framework the table top is commonly provided with a mesh of tennis racquet type supported by a sparser framework. The attenuation can also be limited by using a composite structure with a core of homogeneous low density foam material and a thin layer of very strong fiber, such as carbon fiber, at the surfaces. Typically the fibers are embedded in a matrix of another material, i.e. forming a fiber reinforced material. For example U.S. Pat. No. 3,897,345, issued to Foster, discloses a lightweight table top comprising an outer skin of carbon or graphite fibers enclosing a rigid polyurethane foam core intended to provide low X-ray attenuation. Another example is U.S. Pat. No. 6,904,630, issued to Al-Kassim et al., that discloses a table top device for supporting and positioning a patient in a medical therapy system, wherein a portion of the frame and support system, that is intended to be located within a beam projection area, is formed substantially from non-metal components in order to provide high transmission of the beam. One example of such a portion is of tennis racquet type with a frame of carbon fiber rods, each of which comprise a carbon fiber skin enclosing a homogenous foam core, framing a carbon fiber grid panel.

The main function of the fiber layer in the composite structures mentioned above is to carry the compressive and tensile stresses generated due to flexure caused by the weight of the patient, while the main function of the core is to support and keep the fiber layers apart at a fixed relative position on opposite sides of the core and to resist shear forces, whereby a lightweight construction with high stiffness is obtained.

When the table top of the composite structures mentioned above is under load from a patient the internal mechanical stress can cause the strong fiber material to delaminate from the core material, primarily due to the limited mechanical properties of the core material, which leads to the core material tearing at or near the interface with the strong fibre material where the forces are greatest and the structure collapsing. This limits the load carrying capability of the table top. Since the mechanical properties and attenuation properties of a given core material are related to the density of the core material it becomes necessary to have a core material with a rather high density in order to fulfill the requirements on rigidity and load carrying capability of the tabletop, but this leads to a higher absorption in the table top than is desired.

### SUMMARY OF THE INVENTION

In view of the foregoing, one object with the present invention is to provide a radiotherapy treatment table top with high rigidity and load carrying capability, while at the same time providing a low attenuation of radiation.

The object is achieved by the table top device as defined in the independent claim. The table top device comprises a foam core at least partly enclosed by a skin of a fiber material, whereby the foam core is sandwiched between skins on opposite sides of the table top device. Preferably the foam core is completely enclosed in a shell formed of upper and lower surface skins, side walls and end walls. The foam core is non-homogenous with respect to density. Preferably the foam core has a varying density, with higher density foam close to the interface of the foam with the upper skin or the interface of the foam with the lower skin or with the interfaces of the foam with the upper skin and the lower skin.

In one embodiment the table top device according to the invention has a foam core with a higher density in an upper layer close to an interface with the skin at the upper side of the table top device (the upper side being the side on which a patient is intended to be supported) where the tension stresses in the core under load are greatest and in a lower layer close to an interface with the skin at the lower side of the table top device where the compression stresses in the core under load are greatest and a lower density towards the center of the foam core where these stresses are lower.

In another embodiment, the table top device comprises a foam core that has a lower density in an upper layer close to an interface with the skin at the upper side of the table top device and a higher density in a lower layer close to an interface with the skin at the lower side of the table top device.

Embodiments of the invention are set forth in the dependent claims. Other objects, advantages and novel features of the invention will become apparent from the following detailed description of the invention when considered in conjunction with the accompanying drawings and claims.

### BRIEF DESCRIPTION OF THE DRAWINGS

Preferred embodiments of the invention will now be described with reference to the accompanying drawings, wherein:

FIG. 1 schematically illustrates a radiotherapy patient support system according to prior art;

FIG. 2a is a schematic cross-sectional view of a table top device having a foam core with high-density outer layers and a low-density central layer according to the present invention, and FIG. 2b is a schematic diagram showing the density variation through the foam core;

FIG. 3a is a schematic cross-sectional view of a section of a table top device according to the present invention under load, and FIG. 3b is a corresponding schematic stress diagram;

FIG. 4a is a schematic cross-sectional view of a table top device having a foam core with a high-density lower layer and a low-density upper layer according to the present invention, and FIG. 4b is a schematic diagram showing the density variation through the foam core;

FIG. 5a is a schematic cross-sectional view of a table top device according to the present invention, and FIGS. 5b-g are schematic diagrams schematically illustrating different density variations in the foam core.

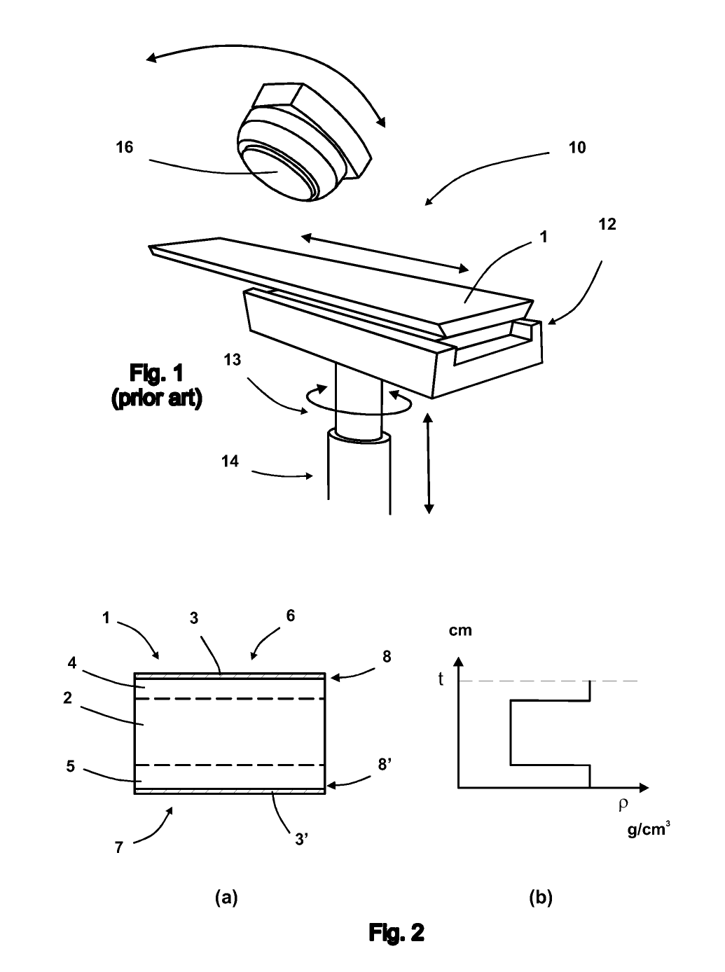

### DETAILED DESCRIPTION OF EMBODIMENTS

Referring to FIG. 2a, a table top device 1 for supporting of a patient in a prescribed position in radiotherapy or diagnostic imaging according to the present invention comprises an elongated core 2 at least partly enclosed between an upper skin 3 and a lower skin 3′, such that the core 2 is sandwiched between the skins 3, 3′ positioned on opposite sides 6, 7 of the table top device 1. Preferably the skins cooperate with side walls and end walls to form a casing which substantially completely encloses the core. The present invention provides a foam core 2 that is non-homogenous with respect to density, ρ, over its vertical thickness, t, which vertical thickness extends in the direction from the interface 8 of the core with the lower skin 3′ to the interface 8′ of the core with the upper skin 3. Preferably the interfaces of the core with the skins are substantially rigid, i.e. the interfaces are in the form of a bond between the core and skins. Vertical thickness t is between 2 and 20 cm, preferably between 3 and 15 cm, more preferably between 4 and 10 cm and most preferably between 5 and 8 cm. The width of a tabletop device is typically between 20 and 60 cm and its length typically 190-260 cm—other dimensions are, of course, possible. Preferably the core 2 has a varying density, with higher density close to at least one interface 4, 8′ with the skin 3, 3′. By way of example, as schematically illustrated in FIGS. 2a-b, the table top device has a polyurethane foam core 2 with a higher density e.g. 0.1 g/cm³ in an upper layer 4 close to an interface with the skin 3 at the upper side 6 of the table top device 1 and in a lower layer 5 close to an interface with the skin 3′ at the lower side 7 of the table top device 1 and a lower density e.g. 0.05 g/cm³ at the center of the core 2. The upper side 6 is the side that the patient is to be placed on. Preferably the core 2 comprises a foamed material and in the following description the terms core and foam core are used interchangeably.

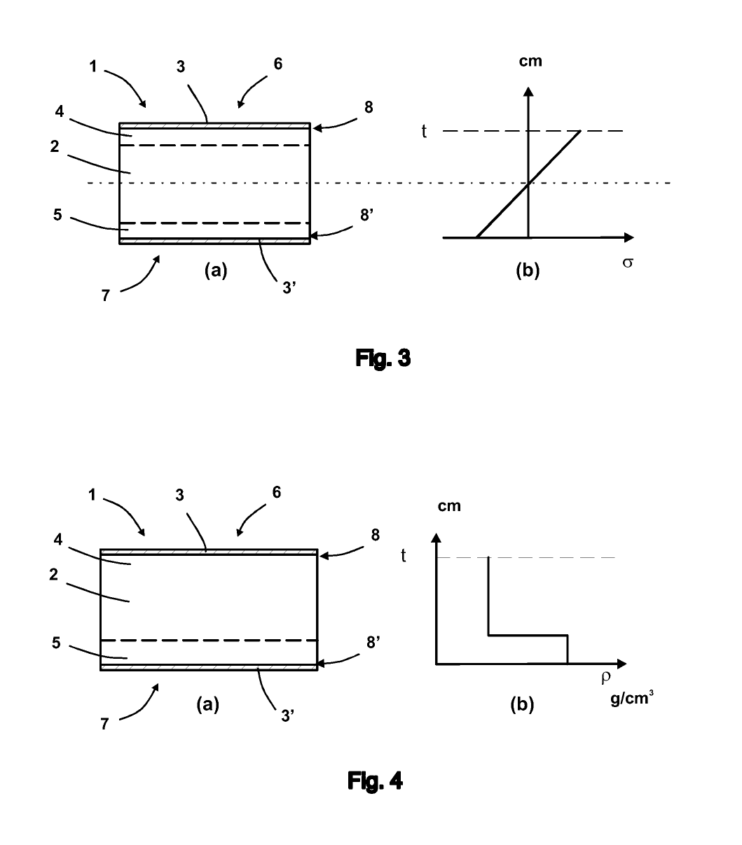

FIG. 3a schematically illustrates a table top device 1 under load. As shown in FIG. 3b the internal mechanical stress, σ, within the foam core 2 vary from compressive stresses at the lower side 7 of the table top device 1 and tensile stresses at the upper side 6 of the table top device 1. From the diagram of FIG. 3b can be seen that the stress levels are higher close to an interface to the skin 3 than in the center of the foam core 2. As mentioned above, by increasing the density of a typical core material the mechanical properties as well as the attenuation properties with regards to the core material are changed. The higher density gives a higher rigidity of the foam core material due to an increased Young's modulus and the tensile strength as well as the shear strength increase, and hence the core material can resist collapse due to shear forces better and better support the skin 3. Since the stress levels are higher close to the interface between the foam core 2 and the skin 3 the advantageous effect of having a higher density is best utilized close to this interface, while a lower density (which gives less attenuation) can be used close to the center of the table top device 1.

Referring to FIGS. 4a-b, in one embodiment of a table top device according to the present invention the foam core 2 has a lower density in an upper layer 4 close to an interface with the skin 3 at the upper side 6 of the table top device 1 and a higher density in a lower layer 5 close to an interface with the skin 3′ at the lower side 7 of the table top device 1. As shown in FIGS. 4a-b the foam core 2 of this embodiment may be a double layer structure, however the present invention is not limited to this. For example, in an alternative embodiment, the table top device 1 may comprise one or more intermediate layers, preferably each having a lower density than the surrounding layers which are closer to a skin. As mentioned above, the lower side 7 of the table top device 1 is subjected to compression and the upper side 6 is subjected to tension. Consequently the stress situation is different at the upper and lower sides 6, 7, respectively, and by having different foam densities in the upper and lower layers 4,5 the stiffness and load carrying capability can be improved while providing low radiation attenuation.

The density variation of the foam core 2 can be achieved by laminating together layers of materials having different densities e.g. by welding, gluing or co-extrusion or by modulating the properties of pre-fabricated core material. The modulation may comprise the step of applying appropriate pressure and heat to the pre-fabricated foam core in order to obtain a material at the surface of the pre-fabricated foam core which has a higher density that the material nearer the centre of the core.

The skins 3, 3′ on the upper side 6 and the lower side 7 of the table top device 1 may have different properties with respect to composition or thickness in order to adapt them to the different requirements on the different sides of the table top device. Under load from a patient the skin 3 on the upper side 6 of the table top device 1 is subjected to tensile stress and the skin 3′ on the lower side 7 of the table top device is subjected to compressive stress. Hence, the required mechanical properties of the skins 3 on the opposite sides are different. In one embodiment of the present invention where the skins are made of fibre reinforced material the lower skin 3′ (which is under compression) is thicker than the upper skin 3 (which is under tension) as fibre reinforced material generally has greater strength under tension than compression, i.e. is better able to resist tensile stresses than compressive stresses.

Skins 3, 3′ are preferably made of a material comprising fibers, for example fiber reinforced composite material. In one embodiment the skins 3, 3′ comprises carbon fibers, and in another embodiment the skins 3, 3′ comprises aramid fibers. One advantage with aramid fibers over carbon fibers is that they enable magnetic resonance imaging. The carbon fibers have a shielding effect that affects the magnetic resonance imaging. Also fiber reinforced polymers may be used. One example of such is a carbon fiber reinforced epoxy. Mixtures of fibers are also conceivable. Typically, the mechanical properties, such as tensile strength, shear strength and Young's modulus, of the skin are superior to the mechanical properties of the core. The fibers of the skin 3 can be provided in a disordered manner, as a woven fabric, or orderly arranged in some other way, for example with a majority of the fibres arranged in the direction of greatest stress when under load e.g. in the longitudinal direction of the table top device.

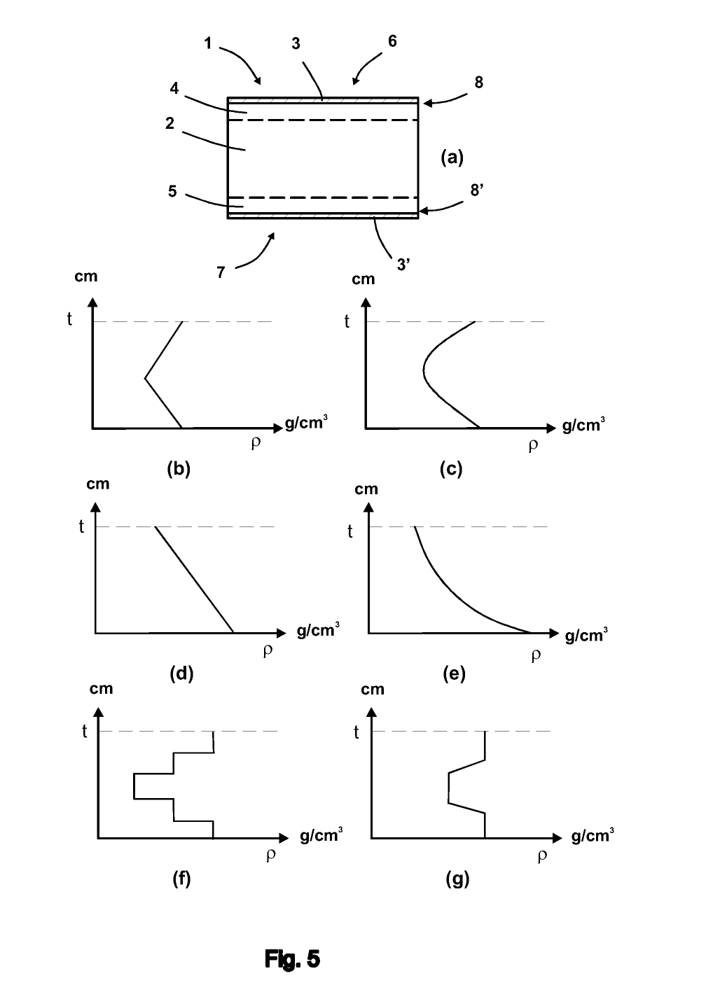

The density variation can be accomplished by having a stepwise change between two layers of the foam core 2 or with a gradual change in density. A smooth gradual change in density can be achieved in the core material manufacturing process or a step-wise gradual density change can be achieved by laminating several sub-layers of core material with different densities in a pile to form a core with the desired configuration. In general, it is preferable when the density is decreased in a direction from the skin 3 towards the central plane parallel to the upper and lower sides of the core 2. FIG. 5b-g schematically illustrates alternative embodiments with different density variation profiles of the foam core 2 of the table top device 1 in FIG. 5a. In FIG. 5b the density decrease substantially linearly from the interfaces between the skin 3,3′ and the foam core 2 at the upper and lower sides 5,6 towards the central plane of the foam core 2. In FIG. 5c the density variation towards the central plane of the foam core 2 is of a quadratic nature. As illustrated in FIGS. 5d and e, the change in density between two layers does not have to be abrupt, but can be smooth e.g. as a result of modulation of the density of the foam core material. FIG. 5f illustrates a step-wise decrease of the density towards the central plane of the foam core, which can be achieved by joining together a plurality of layers with different densities. As illustrated in FIG. 5g, the transition from one density level to another can be gradual. These are alternative embodiments of the double layer approach described above.

The core 2 can be made of one or more core materials in order to achieve the desired variation in density. Preferably, the core comprises one or more polymer foams, such as polyurethane foam. The density of such materials can be varied within a wide range. For example the density of polyurethane can be varied with a factor of about 100, i.e. the density can be varied from about 0.01 to 1 g/cm³. Preferably the density of the foam core 2 when polyurethane is used is within the range from 0.02 to 0.1 g/cm³, in order to limit the attenuation of radiation. Again taking polyurethane as an example, the tensile strength, the shear strength and the Young's modulus of a polyurethane foam are all significantly increased when the density increases. The mechanical properties improve with up to a factor of 10 over the full range of potential density variation. Other polymer foam core materials suitable for the foam core 2 of the invention are polyvinyl chloride (PVC), polystyrene (PS), polymethyl methacrylimide (acrylic), polyetherimide (PEI) and styrenacrylonitrile (SAN), however the invention is not limited to these. Typically these foam core materials are available in densities ranging from 0.03 to more than 0.3 g/cm³, however this range can be extended. As new techniques develop for manufacturing polymer foams, new foam core materials suitable for the foam core 2 of this invention become available. While the invention has been exemplified by polymer foams cores it is appreciated by a person skilled in the art that the invention is not limited to this, but also other foam-like materials such as wood or cellulose-based materials can be used.

The core-skin structure of the table top device 1 of the present invention may be utilized to make the whole table top or only a portion thereof. For example the core-skin structure of the present invention can be used only in the beam projection area and a conventional table top construction used in the remaining part of the table top. In another example a table top device according to the invention is partly supported by an underlying rigid structure and is cantilevered only in the beam projection area. In one implementation of a table top device according to the present invention a 6 cm thick polyurethane foam core 2 is sandwiched between a 0.4 mm thick skin 3 made of Kevlar reinforced composite material at the upper side 6 of table top device 1 and a 1 mm thick skin 3′ made of Kevlar reinforced composite material at the lower side 7 of the table top device. The density of the foam core 2 in a 1 cm thick upper layer 4 and a 1 cm thick lower layer 5 close to the skins 3, 3′ is 0.1 g/cm³ and the density of the remaining part of the foam core 2 between the upper and lower layers 4, 5 is 0.05 g/cm³. The dimensions and properties of this table top device are suitable for forming a 2 m long and 50 cm wide table top for radiation therapy or diagnostic imaging. With such a table top a patient with a weight of up to 200 kg can be safely supported in a prescribed position during treatment.

For the description of the present invention the term skin was used for the enclosing layer of the table top device. Commonly the term shell is used for the same feature. Often the term skin implies that the enclosing layer has been applied to a pre-fabricated foam core and the term shell implies that the enclosing layer has been filled with the foam in order to form the foam core within the enclosing layer. However, the present invention is not limited to either of these approaches.

While the invention has been described in connection with what is presently considered to be the most practical and preferred embodiments, it is to be understood that the invention is not to be limited to the disclosed embodiments, on the contrary, it is intended to cover various modifications and equivalent arrangements within the scope of the appended claims.

1. A table top device (1) for radiotherapy and diagnostic imaging comprising a elongated core (2) of thickness t sandwiched by upper and lower skins (3,3′) arranged on opposite sides (6,7) of the core (2) and joined to said upper skin (3) by an upper interface (8) and joined to lower skin (3′) by a lower interface (8′), characterized in that the core (2) has a varying density in the direction of its thickness t with a higher density close to at least one interface (8, 8′) of the core (2) with a skin (3, 3′).

2. The table top device according to claim 1, wherein said core (2) comprises a foamed material.

3. The table top device according to claim 1, wherein said skins (3,3′) comprise reinforcing fibers.

4. The table top device (1) according to claim 1, wherein the material of said core (2) has a higher density in an upper layer (4) close to the upper interface (8) with the skin (3) and in a lower layer (5) close to the lower interface (8′) with the skin (3′) and a lower density towards the central plane of the core (2).

5. The table top device (1) according to claim 1, wherein the material of the core (2) has a lower density in an upper layer (4) close to the interface (8) with the skin (3) and a higher density in a lower layer (5) close to the lower interface with the skin (3′).

6. The table top device (1) according to claim 1, wherein the material of said core (2) has a gradual decrease in density in a direction from said at least one interface with the skin (3, 3′) towards the center of the core (2).

7. The table top device (1) according to claim 6, wherein the core (2) comprises a plurality of layers with different densities.

8. The table top device (1) according to claim 1, wherein the core (2) comprises polyurethane and/or polymethyl methacrylimide foam.

9. The table top device (1) according to claim 1, wherein the skin (3′) at the lower side (7) is thicker than on the skin (3) on the upper side (6).

10. The table top device (1) according to claim 3, wherein a skin (3,3′) comprises carbon fibers.

11. The table top device (1) according to claim 3, wherein a skin comprises aramid fibers.

12. The table top device according to claim 2, wherein said skins (3,3′) comprise reinforcing fibers.

13. The table top device (1) according to claim 2, wherein the skin (3′) at the lower side (7) is thicker than on the skin (3) on the upper side (6).

14. The table top device (1) according to claim 3, wherein the skin (3′) at the lower side (7) is thicker than on the skin (3) on the upper side (6).
